(19) United States
(12) Patent Application Publication
(10) Pub. No.: US 2008/0154292 A1
(43) Pub. Date: Jun. 26, 2008

(54) Method of operating a microsurgical instrument

(76) Inventors: John C. Huculak, Mission Viejo, CA (US); Bruno Dacquay, Irvine, CA (US)

Correspondence Address: ALCON, IP LEGAL, TB4-8, 6201 SOUTH FREEWAY, FORT WORTH, TX, 76134, US

(21) Appl. No.: 11/935,467

(22) Filed: Nov. 6, 2007

Related U.S. Application Data
(60) Provisional application No. 60/871,467, filed on Dec. 22, 2006.

(51) Int. Cl. A61B 17/32

(52) U.S. Cl. 606/167

(57) ABSTRACT

A method of operating a microsurgical instrument by varying cut rate, port open duty cycle, or both cut rate and port open duty cycle in response to a fluidic signal.

This application claims the priority of U.S. Provisional Application No. 60/871,467 filed Dec. 22, 2006.

### FIELD OF THE INVENTION

The present invention generally pertains to a method of operating microsurgical instruments. More particularly, but not by way of limitation, the present invention pertains to a method of operating microsurgical instruments used in posterior segment ophthalmic surgery, such as vitrectomy probes.

### DESCRIPTION OF THE RELATED ART

Many microsurgical procedures require precision cutting and/or removal of various body tissues. For example, certain ophthalmic surgical procedures require the cutting and/or removal of the vitreous humor, a transparent jelly-like material that fills the posterior segment of the eye. The vitreous humor, or vitreous, is composed of numerous microscopic fibers that are often attached to the retina. Therefore, cutting and removal of the vitreous must be done with great care to avoid traction on the retina, the separation of the retina from the choroid, a retinal tear, or, in the worst case, cutting and removal of the retina itself.

The use of microsurgical cutting probes in posterior segment ophthalmic surgery is well known. Such vitrectomy probes are typically inserted via an incision in the sclera near the pars plana. The surgeon may also insert other microsurgical instruments such as a fiber optic illuminator, an infusion cannula, or an aspiration probe during the posterior segment surgery. The surgeon performs the procedure while viewing the eye under a microscope.

Conventional vitrectomy probes typically include a hollow outer cutting member, a hollow inner cutting member arranged coaxially with and movably disposed within the hollow outer cutting member, and a port extending radially through the outer cutting member near the distal end thereof. Vitreous humor is aspirated into the open port, and the inner member is actuated, closing the port. Upon the closing of the port, cutting surfaces on both the inner and outer cutting members cooperate to cut the vitreous, and the cut vitreous is then aspirated away through the inner cutting member. U.S. Pat. Nos. 4,577,629 (Martinez); U.S. Pat. No. 5,019,035 (Missirlian et al.); U.S. Pat. No. 4,909,249 (Akkas et al.); U.S. Pat. No. 5,176,628 (Charles et al.); U.S. Pat. No. 5,047,008 (de Juan et al.); U.S. Pat. No. 4,696,298 (Higgins et al.); and U.S. Pat. No. 5,733,297 (Wang) all disclose various types of vitrectomy probes, and each of these patents is incorporated herein in its entirety by reference.

Conventional vitrectomy probes include “guillotine style” probes and rotational probes. A guillotine style probe has an inner cutting member that reciprocates along its longitudinal axis. A rotational probe has an inner cutting member that reciprocates around its longitudinal axis. In both types of probes, the inner cutting members are actuated using various methods. For example, the inner cutting member can be moved from the open port position to the closed port position by pneumatic pressure against a piston or diaphragm assembly that overcomes a mechanical spring. Upon removal of the pneumatic pressure, the spring returns the inner cutting member from the closed port position to the open port position. As another example, the inner cutting member can be moved from the open port position to the closed port position using a first source of pneumatic pressure, and then can be moved from the closed port position to the open port position using a second source of pneumatic pressure. As a further example, the inner cutting member can be electromechanically actuated between the open and closed port positions using a conventional rotating electric motor or a solenoid. U.S. Pat. No. 4,577,629 provides an example of a guillotine style, pneumatic piston/mechanical spring actuated probe. U.S. Pat. Nos. 4,909,249 and 5,019,035 disclose guillotine style, pneumatic diaphragm/mechanical spring actuated probes. U.S. Pat. No. 5,176,628 shows a rotational dual pneumatic drive probe.

With each of the above-described vitrectomy probes, the inner cutting member is actuated, and thus the port is opened and closed, over a range of cycle or cut rates. A foot controller is often utilized to allow a surgeon to proportionally control such cycle or cut rate. In addition, the surgeon may have to instruct a nurse how to alter additional surgical parameters (e.g. aspiration vacuum level, aspiration flow rate) on the surgical console to which the vitrectomy probe is operatively attached, or use more complicated foot controllers to alter such parameters, during the surgery. Controlling multiple surgical parameters makes the surgery more complex for the surgeon. Therefore, a need remains for simplified methods of operating a vitrectomy probe or other microsurgical instrument that maximize patient safety.

### SUMMARY OF THE INVENTION

The present invention is a method of operating a microsurgical instrument coupled to a microsurgical system. The instrument includes a port for receiving tissue and an inner cutting member. A flow of tissue is induced into the port with a vacuum source. The inner cutting member is actuated to close the port and cut the tissue. A fluidic signal is provided, and the cut rate of the inner cutting member, the port open duty cycle of the instrument, or both the cut rate of the inner cutting member and the port open duty cycle of the instrument are varied in response to the fluidic signal.

### BRIEF DESCRIPTION OF THE DRAWINGS

For a more complete understanding of the present invention, and for further objects and advantages thereof, reference is made to the following description taken in conjunction with the accompanying drawings in which:

FIG. 1 is a side sectional view of a first vitrectomy probe preferred for use in the method of the present invention shown in the fully open port position;

FIG. 2 is a side sectional view of the probe of FIG. 1 shown in a closed port position;

FIG. 3 is a side, partially sectional view of a second vitrectomy probe preferred for use in the method of the present invention shown in a fully open port position;

FIG. 4 is a cross-sectional view of the probe of FIG. 3 along line 4-4;

FIG. 5 is a cross-sectional view of the probe of FIG. 3 along line 4-4 shown in a closed port position;

FIG. 6 is a block diagram of certain portions of a microsurgical system preferred for use in the method of the present invention;

FIG. 7 is a side sectional view of the probe of FIG. 1 with its port occluded by tissue;

FIG. 8 is an exemplary electrical signal diagram for creating a pneumatic waveform for operation of the probe of FIG. 1; and

FIG. 9 is an exemplary pneumatic waveform for operation of the probe of FIG. 1.

### DETAILED DESCRIPTION OF THE PREFERRED EMBODIMENTS

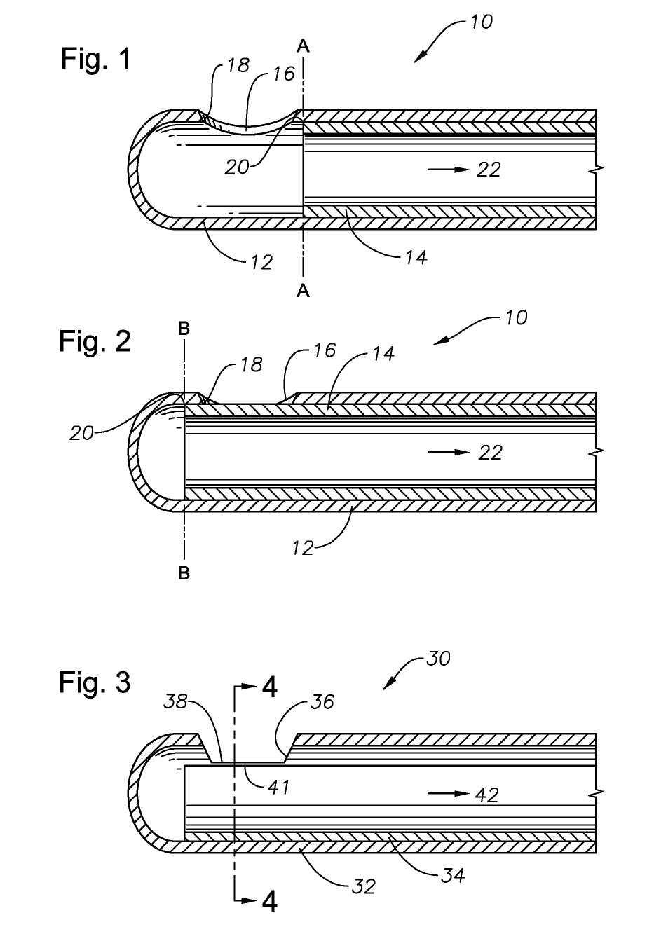

The preferred embodiments of the present invention and their advantages are best understood by referring to FIGS. 1 through 9 of the drawings, like numerals being used for like and corresponding parts of the various drawings.

Referring first to FIGS. 1 and 2, a distal end of a microsurgical instrument 10 is schematically illustrated. Microsurgical instrument 10 is preferably a guillotine style vitrectomy probe and includes a tubular outer cutting member 12 and a tubular inner cutting member 14 movably disposed within outer cutting member 12. Outer cutting member 12 has a port 16 and a cutting edge 18. Inner cutting member 14 has a cutting edge 20.

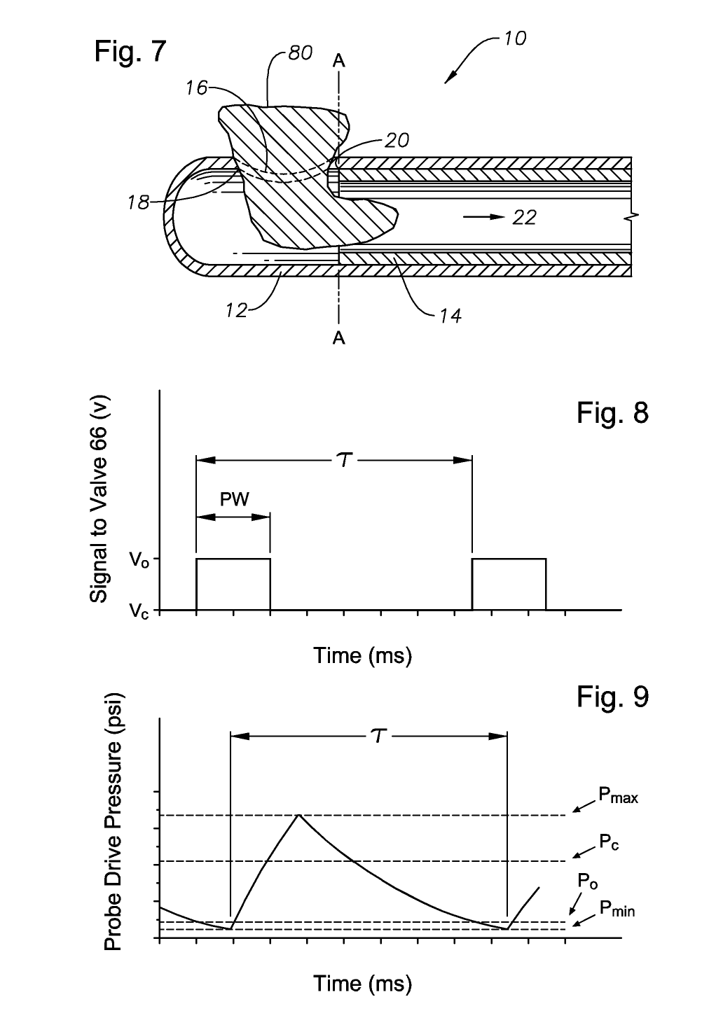

During operation of probe 10, inner cutting member 14 is moved along the longitudinal axis of probe 10 from a position A as shown in FIG. 1, to a position B as shown in FIG. 2, and then back to position A in a single cut cycle. Position A corresponds to a fully open position of port 16, and position B corresponds to a fully closed position of port 16. In position A, vitreous humor or other tissue 80 is aspirated into port 16 and within inner cutting member 14 by vacuum induced fluid flow represented by arrow 22, as shown best in FIG. 7. In position B, the vitreous within port 16 and inner cutting member 14 is cut or severed by cutting edges 18 and 20 and is aspirated away by vacuum induced fluid flow 22. Cutting edges 18 and 20 are preferably formed in an interference fit to insure cutting of the vitreous. In addition, positions A and B may be located somewhat outside the ends of port 16 to account for variations in the actuation of inner cutting member 14 in specific probes 10.

Referring now to FIGS. 3 through 5, a distal end of a microsurgical instrument 30 is schematically illustrated. Instrument 30 is preferably a rotational vitrectomy probe and includes a tubular outer cutting member 32 and a tubular inner cutting member 34 movably disposed within outer cutting member 32. Outer cutting member 32 has a port 36 and a cutting edge 38. Inner cutting member 34 has an opening 40 having a cutting edge 41.

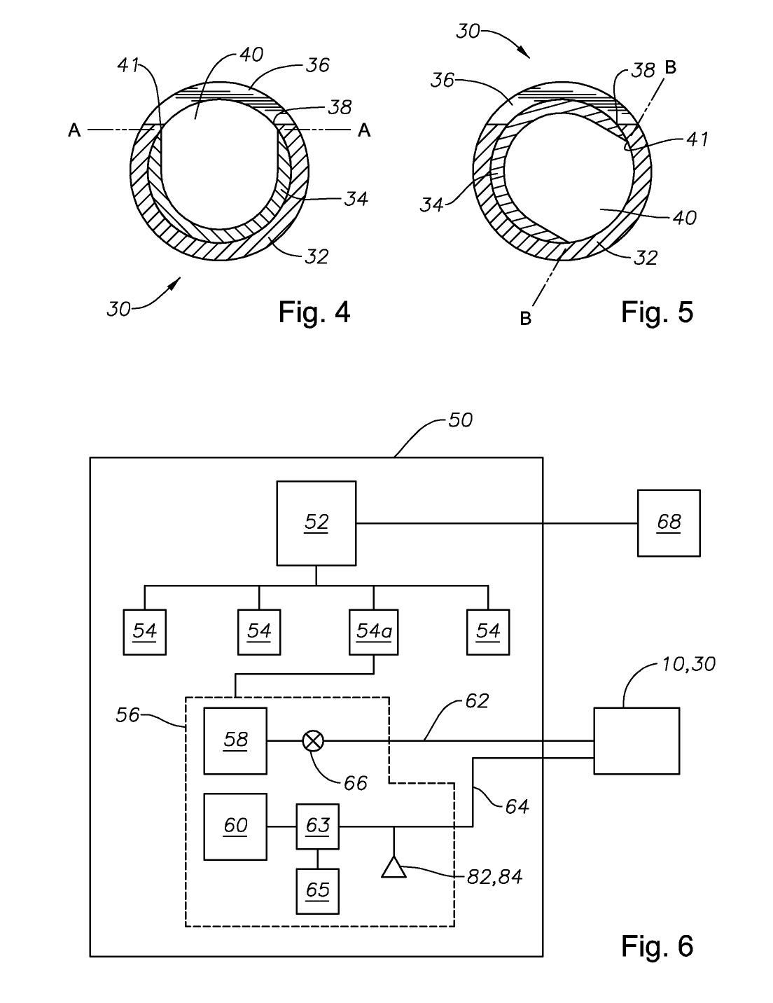

During operation of probe 30, inner cutting member 34 is rotated about the longitudinal axis of probe 30 from a position A as shown in FIG. 4, to a position B as shown in FIG. 5, and then back to position A in a single cut cycle. Position A corresponds to a fully open position of port 36, and position B corresponds to a fully closed position of port 36. In position A, vitreous humor or other tissue is aspirated into port 36, opening 40, and inner cutting member 34 by vacuum induced fluid flow represented by arrow 42. In position B, the vitreous within inner cutting member 34 is cut or severed by cutting edges 38 and 41 and is aspirated away by vacuum induced flow 42. Cutting edges 38 and 41 are preferably formed in an interference fit to insure cutting of the vitreous. In addition, position B may be located somewhat past the edge of cutting surface 38 of outer cutting member 32 to account for variations in the actuation of inner cutting member 34 in specific probes 30.

Inner cutting member 14 of probe 10 is preferably moved from the open port position to the closed port position by application of pneumatic pressure against a piston or diaphragm assembly that overcomes a mechanical spring. Upon removal of the pneumatic pressure, the spring returns inner cutting member 14 from the closed port position to the open port position. Inner cutting member 34 of probe 20 is preferably moved from the open port position to the closed port position using a first source of pneumatic pressure, and then moved from the closed port position to the open port position using a second source of pneumatic pressure. Alternatively, inner cutting members 14 and 34 can be electromechanically actuated between their respective open and closed port positions using a conventional linear motor or solenoid. The implementation of certain ones of these actuation methods is more fully described in U.S. Pat. Nos. 4,577,629; 4,909,249; 5,019,035; and 5,176,628 mentioned above. For purposes of illustration and not by way of limitation, the method of the present invention will be described hereinafter with reference to a guillotine style, pneumatic/mechanical spring actuated vitrectomy probe 10.

FIG. 6 shows a block diagram of certain portions of an electronic and pneumatic sub-assemblies of a microsurgical system 50 preferred for use in the present invention. System 50 preferably includes a host microcomputer 52 that is electronically connected to a plurality of microcontrollers 54. Microcontroller 54a is electronically connected with and controls an air/fluid module 56 of system 50. Air/fluid module 56 preferably includes a source of pneumatic pressure 58 and a source of vacuum 60, both of which are in fluid communication with probe 10 or probe 30 via PVC tubing 62 and 64. Vacuum source 60 preferably comprises a venturi coupled to a pneumatic pressure source. Alternatively, vacuum source 60 may include a positive displacement pump, such as a peristaltic, diaphragm, centrifugal, or scroll pump, or another conventional source of vacuum. A surgical cassette 63 is preferably disposed between aspiration line 64 and vacuum source 60. A collection bag 65 is preferably fluidly coupled to cassette 63 for the collection of aspirated tissue and other fluid from the eye. Air/fluid module 56 also preferably includes appropriate electrical connections between its various components. Although both probes 10 and 30 may be used with system 50, the remainder of this description of system 50 will only reference probe 10 for ease of description.

Pneumatic pressure source 58 provides pneumatic drive pressure to probe 10. A solenoid valve 66 is disposed within tubing 62 between pneumatic pressure source 58 and probe 10. System 50 also preferably includes a variable controller 68. Variable controller 68 is preferably electronically connected with and controls solenoid valve 66 via microcomputer 52 and microcontroller 54a. In this mode of operation, variable controller 68 provides a variable electric signal that cycles solenoid valve 66 between open and closed positions so as to provide a cycled pneumatic pressure that drives inner cutting member 14 of probe 10 from its open port position to its closed port position at a variety of cut rates. Although not shown in FIG. 6, air/fluid module 56 may also include a second pneumatic pressure source and solenoid valve controlled by microcontroller 54a that drives inner cutting member 34 of probe 30 from its closed port position to its open port position. Variable controller 68 is preferably a foot switch or foot pedal that is operable by a surgeon. Alternatively, variable controller 68 could also be a hand held switch or “touch screen” control, if desired.

Microcomputer 52 may also provide an additional control signal or signals to microcontroller 54a indicative of the calculated intraocular pressure of the patient, the measured or calculated aspiration vacuum within the aspiration circuit of microsurgical system 50, the measured or calculated aspiration flow rate within the aspiration circuit of microsurgical system 50, or a combination of one or more of such surgical parameters. As used in this document, such signals shall be collectively referred to as “fluidic signals”. A flow meter 82, pressure transducer 84, or other conventional sensor may be used to measure such aspiration flow rate or aspiration vacuum, respectively. In addition, U.S. application Ser. No. 11/158,238 filed Jun. 21, 2005 and Ser. No. 11/158,259 filed Jun. 21, 2005, which are incorporated herein by reference, more fully describe methods of calculating aspiration flow rate. U.S. application Ser. No. 11/237,503 filed Sep. 28, 2005, which is incorporated herein by reference, more fully describes methods of calculating intraocular pressure. Microcomputer 52 and microcontroller 54a may utilize the fluidic signal or signals to cycle solenoid valve 66 between open and closed positions so as to control the cut rate of probe 10.

Referring to FIG. 8, an exemplary electrical signal supplied by microcontroller 54a to solenoid valve 66 so as to actuate inner cutting member 14 of probe 10 via pneumatic pressure source 58 and tubing 62 is shown. The closed position of valve 66 is preferably assigned a value of V_{c}, and the open position of valve 66 is preferably assigned a value of Vₒ. For a given cut rate, probe 10 will have a period τ representative of the time to open valve 66, plus the time valve 66 is held open, plus the time to close valve 66, plus the time valve 66 is held closed until the next signal to open valve 66 occurs. τ is the inverse of cut rate. For the purposes of this document, the duration of the electrical signal that holds valve 66 in the open position is defined as the pulse width PW. As used in this document, port open duty cycle, or duty cycle, is defined as the ratio of PW to τ (PW/τ).

Referring to FIG. 9, τ also represents the time between respective pneumatic pulses generated by air/fluid module 56 in response to the electrical signal of FIG. 8. Pressure Pc represents the pressure at a fully closed port position B, and pressure Po represents the pressure at a fully open port position B. Each pressure pulse has a maximum pressure Pmax and a minimum pressure Pmin. Pc, Po, Pmax, and Pmin may vary for different probes.

In order to accomplish different surgical objectives, it may be desirable to vary the port open duty cycle of probe 10 over a range of cut rates. Microcomputer 52 and microcontroller 54a may also utilize the fluidic signal or signals to vary PW so as to control the port open duty cycle.

Although the preferred method of operation of a microsurgical instrument has been described above with reference to a pneumatic/mechanical spring actuated probe 10, it will be apparent to one skilled in the art that it is equally applicable to a dual pneumatically actuated probe 30. In addition, the preferred method is also applicable to vitrectomy probes that are actuated using a conventional linear electrical motor, solenoid, or other electromechanical apparatus.

From the above, it may be appreciated that the present invention provides an improved method of operating a vitrectomy probe or other microsurgical cutting instrument. The improved method is simple for the surgeon and safe for the patient.

It is believed that the operation and construction of the present invention will be apparent from the foregoing description. While the apparatus and methods shown or described above have been characterized as being preferred, various changes and modifications may be made therein without departing from the spirit and scope of the invention as defined in the following claims.

What is claimed is:

1. A method of operating a microsurgical instrument coupled to a microsurgical system, said instrument comprising a port for receiving tissue and an inner cutting member, comprising the steps of:
inducing a flow of tissue into said port with a vacuum source;
actuating said inner cutting member to close said port and cut said tissue;
providing a fluidic signal; and
varying a cut rate of said inner cutting member in response to said fluidic signal.

2. The method of claim 1 wherein said fluidic signal is indicative of a calculated intraocular pressure.

3. The method of claim 1 wherein said fluidic signal is indicative of a measured aspiration vacuum of an aspiration circuit of said microsurgical system.

4. The method of claim 1 wherein said fluidic signal is indicative of a calculated aspiration vacuum of an aspiration circuit of said microsurgical system.

5. The method of claim 1 wherein said fluidic signal is indicative of a measured aspiration flow rate of an aspiration circuit of said microsurgical system.

6. The method of claim 1 wherein said fluidic signal is indicative of a calculated aspiration flow rate of an aspiration circuit of said microsurgical system.

7. The method of claim 1 further comprising the step of varying a port open duty cycle of said instrument in response to said fluidic signal.

8. A method of operating a microsurgical instrument coupled to a microsurgical system, said instrument comprising a port for receiving tissue and an inner cutting member, comprising the steps of:
inducing a flow of tissue into said port with a vacuum source;
actuating said inner cutting member to close said port and cut said tissue;
providing a fluidic signal; and
varying a port open duty cycle of said instrument in response to said fluidic signal.

9. The method of claim 8 wherein said fluidic signal is indicative of a calculated intraocular pressure.

10. The method of claim 8 wherein said fluidic signal is indicative of a measured aspiration vacuum of an aspiration circuit of said microsurgical system.

11. The method of claim 8 wherein said fluidic signal is indicative of a calculated aspiration vacuum of an aspiration circuit of said microsurgical system.

12. The method of claim 8 wherein said fluidic signal is indicative of a measured aspiration flow rate of an aspiration circuit of said microsurgical system.

13. The method of claim 8 wherein said fluidic signal is indicative of a calculated aspiration flow rate of an aspiration circuit of said microsurgical system.
